(19) United States
(12) Patent Application Publication
(10) Pub. No.: US 2015/0051232 A1
(43) Pub. Date: Feb. 19, 2015

(54) USE OF (RS)-S-CYCLOPROPYL-S-(4--5-(TRIFLUOROMETHYL)PYRIMIDIN-2-YL]AMINO}PHENYL)SULFOXIMIDE FOR TREATING SPECIFIC TUMOURS

(71) Applicant: Bayer Intellectual Property GmbH, Monheim (DE)

(72) Inventor: Martin Kornacker, Berlin (DE)

(21) Appl. No.: 14/387,075

(22) PCT Filed: Mar. 18, 2013
(86) PCT No.: PCT/EP2013/055561

§ 371 Date: Sep. 22, 2014

(30) Foreign Application Priority Data
Mar. 21, 2012 (DE) 10 2012 204 506.6

(51) Int. Cl. A61K 31/513

(52) U.S. Cl. CPC A61K 31/513
USPC 514/272

(57) ABSTRACT

The invention relates to the use of (R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide and/or (S)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide for the treatment of specific tumours.

The present invention relates to the use of (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide, in particular (R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide, for the treatment of specific tumours.

The cyclin-dependent kinases (CDKs) are an enzyme family which playes an important role in the regulation of the cell cycle and therefore represents a particularly interesting target for the design of small inhibitory molecules. Selective inhibitors of CDKs can be used for treating cancer or other disorders caused by impaired cell proliferation.

Pyrimidines and analogues have already been described as active compounds, for example the 2-anilinopyrimidines as fungicides (DE 4029650) or substituted pyrimidine derivatives for the treatment of neurological or neurodegenerative disorders (WO 99/19305). Most diverse pyrimidine derivatives are described as CDK inhibitors, for example 2-amino-4-substituted pyrimidines (WO 01/14375), purines (WO 99/02162), 5-cyanopyrimidines (WO 02/04429), anilinopyrimidines (WO 00/12486) and 2-hydroxy-3-N,N-dimethylaminopropoxypyrimidines (WO 00/39101).

WO 02/096888 and WO 03/076437 disclose in particular pyrimidine derivatives having CDK-inhibitory activity.

Examples of active sulphoximine compounds are sulphonimidoyl-modified triazoles as fungicides (H. Kawanishi, H. Morimoto, T. Nakano, T. Watanabe, K. Oda, K. Tsujihara, Heterocycles 1998, 49, 181) or arylalkylsulphoximines as herbicides and pesticides (Shell International Research, Ger. P. 2 129 678).

WO 2005/037800 discloses open sulphoximine-substituted anilinopyrimidine derivatives as inhibitors of cyclin-dependent kinases. Examples are given of structures which, in the 5-position of the pyrimidine, are either unsubstituted or substituted by halogen, in particular bromine. None of the specific structures disclosed has a 5-trifluoromethyl substituent.

The novel pan-CDK inhibitors and processes for their preparation are described in the PCT application PCT/EP2009/007247, the disclosure of which is referred to by the present application and which is incorporated into the present application by this reference. (RS)—S-(4-{[4-{[(1R,2R)-2-Hydroxy-1-methylpropyl]oxy}-5-(trifluoromethyl)pyrimidin-2-yl]amino}phenyl)-S-methylsulphoximide is Exemplary compound 1.

PCT/EP2011/054733 relates to the use of a group of pan-CDK inhibitors for various tumour disorders. (RS)—S-Cyclopropyl-S-(4-{[4-{[(1R,2R)-2-hydroxy-1-methylpropyl]oxy}-5-(trifluoromethyl)pyrimidin-2-yl]amino}phenyl)sulphoximide is Exemplary compound 1.

DE102010014427 relates to the combination of the abovementioned group of pan-CDK inhibitors with other tumour therapeutics for various tumour disorders. (RS)—S-Cyclopropyl-S-(4-{[4-{[(1R,2R)-2-hydroxy-1-methylpropyl]oxy}-5-(trifluoromethyl)pyrimidin-2-yl]amino}phenyl)sulphoximide is Exemplary compound 1.

Based on this prior art, it was the object of the present invention to provide compounds for patients suffering from thyroid cancer, mesotheliomas, squamous cell carcinomas of the oesophagus or cholangiocellular carcinomas, which compounds at least stabilize the tumour disorder, without any side-effects that result in a discontinuation of therapy.

This was foreseeable only to a limited extent.

It has now been found that the compound (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide (Compound A), in particular (R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide (Compound A′), in specific tumour types in humans, indeed leads to stabilization, where side-effects occur to an extent which is easily treatable.

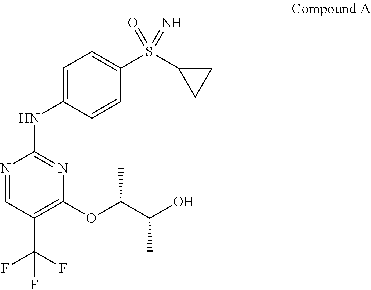 [SMILES: C[C@@H](O)[C@@H](C)Oc1nc(Nc2ccc(S(=N)(=O)C3CC3)cc2)ncc1C(F)(F)F]

(RS)—S-Cyclopropyl-S-(4-{[4-{[(1R,2R)-2-hydroxy-1-methylpropyl]oxy}-5-(trifluoromethyl)pyrimidin-2-yl]amino}phenyl)sulphoximide (Compound A) is a selected sulphoximine-substituted anilinopyrimidine derivative which can be separated into two stereoisomers, namely:
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide (Compound A′) and
(S)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide (Compound A″).

Compound A′ is preferred and is, as BAY1000394, undergoing clinial trials.

If Compound A is referred to hereinbelow, this is understood to mean both the pure stereoisomers A′ and A″ and any mixture of these two compounds.

The present application provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas.

The present application furthermore provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas,

where there are 3 days of treatment and 4 days of non-treatment.

The present application furthermore provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas,

where during the treatment days a dose of from 0.5 mg to 20 mg a day, preferably from 1.0 to 15 mg a day, is taken orally.

In the case of a combination therapy with other antihyperproliferative, cytostatic or cytotoxic substances, it may be necessary to reduce the dose.

The present application furthermore provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for preparing a medicament for the treatment of thyroid cancer, mesotheliomas, squamous cell carcinomas of the oesophagus or cholangiocellular carcinomas.

The present application furthermore provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for preparing a medicament for the treatment of thyroid cancer, mesotheliomas, squamous cell carcinomas of the oesophagus or cholangiocellular carcinomas,

where there are 3 days of treatment and 4 days of non-treatment.

The present application furthermore provides the use of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for preparing a medicament for the treatment of thyroid cancer, mesotheliomas, squamous cell carcinomas of the oesophagus or cholangiocellular carcinomas,

where during the treatment days a dose of from 0.5 mg to 20 mg a day, preferably from 1.0 to 15 mg a day, is taken orally.

In the case of a combination therapy with other antihyperproliferative, cytostatic or cytotoxic substances, it may be necessary to reduce the dose.

The present application furthermore provides (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas.

The present application furthermore provides (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas, where there are 3 days of treatment and 4 days of non-treatment.

The present application furthermore provides (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas,

where during the treatment days a dose of from 0.5 mg to 20 mg a day, preferably from 1.0 to 15 mg a day, is taken orally.

In the case of a combination therapy with other antihyperproliferative, cytostatic or cytotoxic substances, it may be necessary to reduce the dose.

The present application furthermore provides medicaments and pharmaceutical formulations comprising
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas.

The present application furthermore provides medicaments and pharmaceutical formulations comprising
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas, where there are 3 days of treatment and 4 days of non-treatment.

The present application furthermore provides medicaments and pharmaceutical formulations comprising
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

for the treatment of thyroid cancer, mesotheliomas, squamous cell carcinomas of the oesophagus or cholangiocellular carcinomas,

where during the treatment days a dose of from 0.5 mg to 20 mg a day, preferably from 1.0 to 15 mg a day, is taken orally.

In the case of a combination therapy with other antihyperproliferative, cytostatic or cytotoxic substances, it may be necessary to reduce the dose.

Both in monotherapy and in combination therapy, there are preferably 3 days of treatment and 4 days of non-treatment.

However, the treatment protocol is, if required, adapted to the individual disease situation of the patient and/or in the combination therapy with the substance or the substances employed in the combination therapy.

The present application furthermore provides combinations of
(RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

in particular
(R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide

with at least one further active compound for the treatment of thyroid cancer, mesotheliomas, squamous cell carcinomas of the oesophagus or cholangiocellular carcinomas.

The present invention also embraces the use of the physiologically acceptable salts of Compound A.

Physiologically acceptable salts of Compound A include acid addition salts of mineral acids, carboxylic acids and sulfonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of Compound A also include salts of customary bases such as, by way of example and preferably, alkali metal salts (for example sodium salts and potassium salts), alkaline earth metal salts (for example calcium salts and magnesium salts) and ammonium salts derived from ammonia or organic amines having 1 to 16 carbon atoms such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention furthermore provides medicaments comprising compound A and at least one or more further active compounds for the treatment and/or prophylaxis of thyroid cancer, mesotheliomas, squamous cell carcinomas of the oesophagus or cholangiocellular carcinomas.

Compound A according to the invention can act systemically and/or locally. To this end, it can be administered in a suitable manner, for example by the oral, parenteral, pulmonal, nasal, sublingual, lingual, buccal, rectal, dermal, transdermal, conjunctival, otic route, or as an implant or stent.

For these administration routes, Compound A can be administered in suitable administration forms.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the compound according to the invention), films/lyophilizates, capsules (for example hard or soft gelatin capsules), coated tablets, granules, pellets, powders, emulsions, suspensions, aerosols or solutions.

Solutions comprising or consisting of solubilisers, surfactants and/or one or more flavourings have been found to be advantageous for Compound A.

Suitable solubilisers are macrogols, in particular macrogol 400.

Suitable surfactants are polysorbates, in particular polysorbate 20.

Suitable flavourings are essential oils, in particular menthol.

The concentration of the pharmaceutical may be from 0.1 mg/ml to 10 mg/ml, preferably from 0.2 mg/ml to 8 mg/ml, particularly preferably from 0.3 mg/ml to 6 mg/ml and most preferably from 0.4 mg/ml to 4 mg/ml.

Examples given are the concentrations 0.2 mg/ml and 4.8 mg/ml.

Tablets comprising or consisting of fillers, disintegrants and/or one or more additives for pressing have also been found to be advantageous for Compound A.

Suitable fillers are polyols such as mannitol, in particular in granulated form, or else cellulose derivatives such as microcrystalline cellulose.

Suitable additives for pressing are stearates, in particular magnesium stearate.

Suitable disintegrants are cellulose derivatives, in particular croscarmellose.

The concentration of the pharmaceutical may be from 0.1 mg/tablet to 10 mg/tablet, preferably from 0.3 mg/tablet to 8 mg/tablet, particularly preferably from 0.4 mg/tablet to 6 mg/tablet and most preferably from 0.5 mg/tablet to 5 mg/tablet.

An example given is the concentration 5 mg/tablet.

Prior to and for formulation into the form of a medicament, Compound A is preferably present in micronized form.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Suitable administration forms for parenteral administration include injection and infusion formulations in the form of solutions, suspensions, emulsions, lyophilisates or sterile powders.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

Compound A can be converted to the administration forms mentioned.

This can be accomplished in a manner known per se by mixing with inert nontoxic pharmaceutically suitable auxiliaries. These auxiliaries include inter alia carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavour and/or odour correctants.

The present invention further provides medicaments which comprise compound A, typically together with one or more inert, nontoxic, pharmaceutically suitable auxiliaries, and for the use thereof for the aforementioned purposes.

The formulation of Compound A into pharmaceutical preparations is carried out in a manner known per se by converting the active compound(s) into the desired administration form using auxiliaries customary in the art of pharmaceutical formulation.

Here, suitable auxiliaries are, for example, carrier substances, fillers, disintegrants, binders, moisturizers, glidants, absorbants and adsorbants, diluents, solvents, cosolvents, emulsifiers, solubilizers, flavour correctants, colorants, preservatives, stabilizers, wetting agents, salts for changing the osmotic pressure or buffer.

Reference should be made to Remington's Pharmaceutical Science, 15th ed. Mack Publishing Company, East Pennsylvania (1980).

The pharmaceutical formulations may be present

in solid form, for example as tablets, coated tablets, pills, suppositories, capsules, transdermal systems or

in semisolid form, for example as ointments, creams, gels, suppositories, emulsions or

in liquid form, for example as solutions, tinctures, suspensions or emulsions.

Auxiliaries for the purpose of the invention can, for example, be salts, saccharides (mono-, di-, tri-, oligo- and/or polysaccharides), proteins, amino acids, peptides, fats, waxes, oils, hydrocarbons and derivatives thereof, where the auxiliaries may be of natural origin or can be obtained synthetically or by partial synthesis.

Suitable for oral or peroral administration are in particular tablets, coated tablets, capsules, pills, powders, granules, pastilles, suspensions, emulsions or solutions.

Suitable for parenteral administration are in particular suspensions, emulsions and especially solutions.

The present invention relates to the use of Compound A, in particular Compound A′, for the therapy of thyroid cancer, mesotheliomas, squamous cell carcinomas of the oesophagus or cholangiocellular carcinomas

Dosage and Treatment Protocol:

Dosage and treatment protocol can and must be varied depending on the type of carcinoma and the treatment target.

In general, the daily dose is from 0.5 mg to 20 mg and may be divided into a plurality of identical or different dosage units, preferably 2.

The preferred daily dose is from 1.0 mg to 15 mg and may be divided into a plurality of identical or different dosage units, preferably 2.

This applies to monotherapy as well as combination therapy with other antihyperproliferative, cytostatic or cytotoxic substances, where in the case of combination therapy it may be necessary to reduce the dose.

Treatment may be carried out over 2 to 60 days, where the treatment is preferably followed by 2 to 30 days of non-treatment.

Successful treatment protocols were 28 days of treatment followed by 14 days of non-treatment, and in particular 3 days of treatment followed by 4 days of non-treatment.

Treatment is successful if there is at least disease stabilization and the side-effects occur to an extent which is easily treatable, but at least easily acceptable.

Disease stabilization in mesothelioma patients could be achieved using a daily dose of 2.4 mg, 9.6 mg and 19.2 mg which was divided into two identical dosage units.

Here, there were 3 days of treatment and 4 days of non-treatment.

Disease stabilization in thyroid cancer patients could be achieved using a daily dose of 0.6 mg which was divided into two identical dosage units.

Here, there were 28 days of treatment and 14 days of non-treatment.

Disease stabilization in thyroid cancer patients could also be achieved using a daily dose of 15 mg which was divided into two dosage units (5 mg in the morning, 10 mg in the evening).

Here, there were 3 days of treatment and 4 days of non-treatment.

Disease stabilization in a patient suffering from squamous cell carcinoma of the oesophagus could be achieved using a daily dose of 1 mg which was divided into two identical dosage units.

Here, there were 28 days of treatment and 14 days of non-treatment.

Disease stabilization in a patient suffering from cholangiocellular carcinoma could be achieved using a daily dose of 10 mg which was divided into two identical dosage units.

Here, there were 3 days of treatment and 4 days of non-treatment.

Compound A may be employed alone or, if required, in combination with one or more other pharmacologically active substances, provided this combination does not lead to unwanted and unacceptable side-effects. Accordingly, the present invention furthermore provides medicaments comprising at least one of the compounds according to the invention and one or more further active compounds, in particular for the treatment and/or prevention of the disorders mentioned above.

For example, Compound A may be combined with known antihyperproliferative, cytostatic or cytotoxic substances for the treatment of cancerous disorders. The combination of the compounds according to the invention with other substances customary for cancer therapy or else with radiotherapy is particularly indicated.

As examples of active compounds suitable for combinations, there may be mentioned:

Abraxane, afinitor, aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulfan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-nl, interferon-alpha-n3, interferon-beta, interferon-gamma-1α, interleukin-2, intron A, iressa, irinotecan, kytril, lapatinib, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, RDEA119, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compound A can be combined with antihyperproliferative agents, which can be, by way of example—without this list being conclusive:

Abraxane, aminoglutethimide, L-asparaginase, azathioprine, 5-azacytidine, bleomycin, busulfan, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenine, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

In a highly promising manner, compound A can also be combined with biological therapeutics such as antibodies (for example Avastin, Rituxan, Erbitux, Herceptin, cetuximab) and recombinant proteins.

It is also possible to achieve positive effects by using compound A in combination with other therapies directed against angiogenesis such as, for example, with Avastin, axitinib, regorafenib, Recentin, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compound A with other cytostatically or cytotoxically active agents:
improved efficacy in slowing the growth of a tumour, in reducing its size or even in the complete elimination thereof, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of tumours;
the achievement of a higher rate of response to the therapy;
a longer survival time of the patient compared with present-day standard therapy.

In addition, the compounds according to the invention can also be used in conjunction with radiotherapy and/or surgical intervention.

Preparation of the Compounds According to the Invention

The preparation of the compounds according to the invention is extensively described in PCT/EP2009/007247, the disclosure of which is referred to by the present application and which is incorporated into the present application by this reference.

PCT/EP2011/066295, the disclosure of which is likewise referred to by the present application and which is incorporated into the present application by this reference, discloses a preparation which is developed further.

### EXAMPLE 1

### Clinical Trial in Patients with Mesotheliomas

Patients:

Patient 1010, age: 66 years, male, epithelioid mesothelioma, progressive disease on study enrollment, systemic pre-therapies with Alimta and cisplatin, gemcitabine, doxorubicin, dekabine

Patient 1016, age: 55 years, female, epithelioid mesothelioma, stage IV, progressive disease on study enrollment, systemic pre-therapies with Alimta and cisplatin, Alimta and carboplatin, Alimta and carboplatin and Avastin, Avastin, Alimta, gemcitabine

Patient 1023, age: 69 years, male, pleuramesothelioma, stage IV, progressive disease on study enrollment, systemic pre-therapies with cisplatin and Alimta

Patient 1024, age: 50 years, male, pleuramesothelioma, stage IV, progressive disease on study enrollment, systemic pre-therapies with cisplatin and Alimta and Avastin, Avastin, carboplatin and Alimta and doxorubicin and dekapine

Administration Form:

Oral

Formulation

Oral solution in two dose strengths, ingredients micronized BAY 1000394 (0.2 mg/ml and 4.8 mg/ml), Levomenthol (synonym: 1-menthol, flavouring), macrogol (400) (synonym: polyethylene glycol (400), solubilizer), polysorbate 20 (surfactant)

Dosage and Treatment Protocol:

Dosage twice a day 1.2 mg (patient 1010), 4.8 mg (patient 1016) and 9.6 mg (patients 1023 and 1024), administration protocol 3 days of treatment and 4 days of non-treatment, long-term treatment until disease progression

Significant Results:

Patient 1010 achieved disease stabilization according to RECIST 1.1 and was treated for about 4 months

Patient 1016 achieved disease stabilization according to RECIST 1.1 and was treated for more than 3 months

Patient 1023 achieved disease stabilization according to RECIST 1.1 and was treated for about 2 months

Patient 1024 achieved disease stabilization according to RECIST 1.1 and was treated for about 2 months

### EXAMPLE 2

### Clinical Trial in Patients with Thyroid Cancer

Patients:

Patient 2006, clinical study 14856, age: 43 years, male, papillary thyroid cancer stage IV, progressive disease on study enrollment, systemic pre-therapies with 17-AAG, Sutent, sorafenib, erlotinib and temsirolimus

Patient 1030, clinical study 14484, age: 52 years, female, thyroid cancer stage IV, progressive disease on study enrollment, systemic pre-therapies with Adriamycin and cisplatin/carboplatin, carboplatin and Taxol, Nexavar, Sutent

Administration Form:

Oral

Formulation

Patient 2006: oral solution, ingredients micronized BAY 1000394 (0.2 mg/ml), levomenthol (synonym: 1-menthol, flavouring) macrogol (400) (synonym: polyethylene glycol (400), solubilizer), polysorbate 20 (surfactant)

Patient 1030: tablet, ingredients BAY 1000394, micronized, 5 mg/tablet, granulated mannitol (filler), microcrystalline cellulose (filler), croscarmellose (disintegrant), magnesium stearate (additive for pressing), red lacquer (coating)

Dosage and Treatment Protocol:

Patient 2006: dosage twice a day 0.3 mg, administration protocol 28 days of treatment and 14 days of non-treatment, long-term treatment until disease progression

Patient 1030: dosage 5 mg in the morning, 10 mg in the evening, administration protocol 3 days of treatment and 4 days of non-treatment, long-term treatment until disease progression

Significant Results:

Patient 2006: the patient achieved disease stabilization according to RECIST 1.1 and was treated for about 2 months

Patient 1030: the patient achieved disease stabilization according to RECIST 1.1 and has been treated for more than 3 months

### EXAMPLE 3

### Clinical Trial in a Patient with Squamous Cell Carcinomas of the Oesophagus

Patient:

Patient 3002, age: 61 years, female, squamous cell carcinoma of the oesophagus stage IV, progressive disease on study enrollment, systemic pre-therapy with cisplatin and Xeloda

Administration Form:

Oral

Formulation

Oral solution, ingredients micronized BAY 1000394 (0.2 mg/ml), levomenthol (synonym: 1-menthol, flavouring) macrogol (400) (synonym: polyethylene glycol (400), solubilizer), polysorbate 20 (surfactant)

Dosage and Treatment Protocol:

Dosage twice a day 0.5 mg, administration protocol 28 days of treatment and 14 days of non-treatment, long-term treatment until disease progression

Significant Results:

The patient achieved disease stabilization according to RECIST 1.1 and was treated for more than 2 months

### EXAMPLE 4

### Clinical Trial in a Patient with Cholangiocellular Carcinoma

Patient:

Patient 1026, age: 62 years, female, cholangiocellular carcinoma stage IV, progressive disease on study enrollment, systemic pre-therapies with cisplatin/oxaliplatin and Gemzar, 5-fluorouracil and folinic acid

Administration Form:

Oral

Formulation

tablet, ingredients BAY 1000394, micronized, 5 mg/tablet, granulated mannitol (filler), microcrystalline cellulose (filler), croscarmellose (disintegrant), magnesium stearate (additive for pressing), red lacquer (coating)

Dosage and Treatment Protocol:

Dosage twice a day 5 mg, administration protocol 3 days of treatment and 4 days of non-treatment, long-term treatment until disease progression

Significant Results:

The patient achieved disease stabilization according to RECIST 1.1 and has been treated for more than 4 months

1. A method for the treatment of thyroid cancer, mesotheliomas, squamous cell carcinomas of the oesophagus or cholangiocellular carcinomas comprising administering to a patient in need thereof an effective amount of (RS)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide.

2. A method for the treatment of thyroid cancer, mesotheliomas, squamous cell carcinomas of the oesophagus or cholangiocellular carcinomas comprising administering to a patient in need thereof an effective amount of (R)—S-cyclopropyl-S-(4-{[4-{[(1R,2R)-2-hydroxy-1-methylpropyl]oxy}-5-(trifluoromethyl)pyrimidin-2-yl]amino}phenyl)sulphoximide.

3. (canceled)

4. (canceled)

5. The method according to claim 1, wherein a mesothelioma is treated.

6. The method according to claim 1, wherein 3 days of treatment is followed by 4 days of non-treatment.

7. The method according to claim 1, wherein a dose of from 1.0 to 15 mg a day is administered orally.

8. (canceled)

9. (canceled)

10. (canceled)

11. (canceled)

12. (canceled)

13. (canceled)

14. (canceled)

15. The method according to claim 1, further comprising administering at least one additional active compound.

16. The method according to claim 2, further comprising administering at least one additional active compound.

17. The method according to claim 2, wherein a mesothelioma is treated.

18. The method according to claim 2, wherein 3 days of treatment is followed by 4 days of non-treatment.

19. The method according to claim 2, wherein a dose of from 1.0 to 15 mg a day is administered orally.
